(19) United States
(12) Patent Application Publication
(10) Pub. No.: US 2001/0047136 A1
(43) Pub. Date: Nov. 29, 2001

(54) In-vivo tissue inspection and sampling

(76) Inventors: Richard A. Domanik, Libertyville, IL (US); Peter P. Gombrich, Chicago, IL (US)

Correspondence Address: MERCHANT & GOULD PC, P.O. BOX 2903, MINNEAPOLIS, MN, 55402-0903, US

(21) Appl. No.: 09/767,206

(22) Filed: Jan. 22, 2001

Related U.S. Application Data
(60) Provisional application No. 60/177,520, filed on Jan. 21, 2000.

(51) Int. Cl. A61B 6/00

(52) U.S. Cl. 600/473; 600/476; 250/362; 250/459.1

(57) ABSTRACT

An in-vivo tissue inspection device provides for increased signal levels and the ability to discriminate between normal and abnormal tissues through the use of an exogenous fluorescent or fluorogenic reagent. The device reduces the costs of in-situ fluorescent measurements for screening and diagnostic purposes by eliminating the need for an imaging endoscope; simplifying the illuminating and detection means used in the device; and reducing the computing power needed for data reduction; reducing the operator skill level required to make quantitative measurements of in-situ fluorescence, and enabling simultaneous sampling of the ectocervix and the endocervical canal.

### RELATED APPLICATION

This application claims the benefit of provisional application Serial No. 60/177,520, filed Jan. 21, 2000 entitled “IN-VIVO TISSUE INSPECTION AND COLLECTION DEVICE”, which application is incorporated by reference herein.

### TECHNICAL FIELD

The invention relates generally to tissue inspection and more specifically to in vivo tissue inspection. More particularly, the invention relates to in-vivo tissue inspection using extrinsic fluorescence. In a particular embodiment, the invention relates to in-vivo tissue inspection using extrinsic fluorescence measured using noncoherent light gathering optics.

### BACKGROUND

The Pap test is widely regarded as one of the most effective screening tests for cervical cancer and displasia, as evidenced by the major mortality rate reductions that occur wherever Pap screening is widely deployed and readily available. Although sample collection and processing for Pap testing can be performed by persons having relatively little specialized training, sample evaluation requires a substantial and highly skilled supporting infrastructure. This limits the deployment of Pap screening to those areas where such an infrastructure is available. Furthermore, it can require days to weeks to process and evaluate a Pap sample.

Thus, the physician must locate and contact the patient to inform the patient of the results and, if abnormalities were detected, to arrange for a follow-up visit. Contacting the patient is a time consuming process that is not always successful. Even if contact is made, only a fraction of those informed of abnormal results return for follow-up and treatment. This is particularly true in public health screening situations where the patient population is generally transient and where logistics frequently preclude a return visit.

A screening test for cervical displasia and cancer that can be performed and evaluated within the time frame of a typical single gynecological examination is needed to increase the availability of this type of testing. Particularly in the public health sector, it is highly desirable that the test be simple enough that it can be performed by paramedical personnel; that the instrumentation, if any, be compact, rugged and reliable; and that the cost per result be minimized.

Most tissues, including cervical tissues, can be made to fluoresce when illuminated with the appropriate wavelengths of light. The characteristics of this fluorescence can indicate the presence of cellular abnormalities including displasia and cancer. The measurement and characterization of tissue autofluorescence is the basis of many devices and methods that have been proposed as alternatives to or replacements for the traditional Pap procedure.

Autofluorescence measurements are made by illuminating the cervix with light of particular spectral characteristics and collecting and analyzing the resulting fluorescent emissions. These emissions arise from many different cellular constituents such as, but not limited to, collagen, elastin, flavins and heme-containing proteins. The fluorescence emissions from these various species are broad and overlap each other, resulting in what amounts to a continuum of emissions.

Furthermore, the fluorescence emissions from one such specie can couple to, and thus excite, fluorescence in another specie, resulting in a tissue autofluorescence spectrum that is very complex. Tissue autofluorescence also tends to be of low intensity, largely because many of the fluorescent species are weak or inefficient emitters that are present at low concentrations. The reported changes in tissue fluorescent emissions that are associated with the presence of cellular abnormalities are subtle, consisting primarily of small changes in emission intensity or emission wavelength distribution.

Thus, measuring tissue autofluorescence is a difficult undertaking, particularly when performed in-situ, as it involves quantitatively detecting small changes in small signals in the presence of many interferences.

These difficulties are typically addressed in several ways. The level of the desired fluorescent emission is maximized by careful selection of the exciting wavelengths and by increasing the excitation intensity to the point where all of the target fluorophores are saturated, i.e., being excited at the maximum possible rate. Highly sensitive detectors coupled to highly discriminating wavelength selection means are used to capture the desired signals while complex signal processing algorithms are used to extract the desired information.

Lasers are the most commonly used light sources for tissue autofluorescence measurements due to their narrow spectral bandwidths and the high power densities that can be achieved. High intensity arc lamps coupled to an appropriate wavelength selection means are also used for this purpose. These light sources tend to be large, delicate and expensive units that require considerable operator attention during use. Tissue auto fluorescence is typically excited using light in the violet and ultraviolet spectral regions. Light in these spectral regions is known to have the potential to cause tissue damage, especially at the high power densities required in order to obtain the maximum possible signal level, and can pose a hazard to both the operator and the patient.

Autofluorescence measurements generally use photomultiplier tubes, avalanche diodes or intensified array-type imaging detectors (such as a CCD) as the light detection means. Detector selection is based upon the number of wavelengths at which measurements are to be made as well as the spatial and spectral resolution requirements of the particular embodiment. Interference filters are commonly used as the wavelength selection means in cases where only a few wavelengths are of interest while diffraction gratings are typically used when it is desired to acquire data at a large number of wavelengths. As is the case of the light source, these detection assemblies tend to be large, delicate and expensive.

The large number of complex calculations required to extract the desired information from the acquired data dictates that substantial computational power be provided. Suitable computers, yet again, tend to be large and expensive.

Because the light source, detector and signal processing means in a tissue autofluorescence measuring system are large and the cervix to be examined is located in a confined space, the system elements are generally located remotely from the cervix and an endoscope or similar device is used to deliver the exciting light to, and collect the fluorescent emissions from the cervix.

Several limitations of the present art derive from the endoscope that typically is used to transport light from the light source to the cervix and from the cervix to the detector. These endoscopes are constructed as bundles having thousands of individual optical fibers. Lenses and other optical components are attached to each end of the bundle to provide for imaging and other functions. The fiber bundles used in these endoscopes are “coherent”, meaning that the position of a particular fiber within the array of fibers at one end of the bundle being identical to the position of this same fiber within the array of fibers at the opposite end of the bundle. This spatial coherence allows the bundle to transmit a recognizable image from one end of the bundle to the other. Building a coherent fiber bundle is a painstaking task that greatly contributes to the high cost of an endoscope.

Each fiber within the bundle consists of a core (through which light is transmitted) that is surrounded by a cladding that serves to contain the light within the core and to provide some measure of physical protection for the core. Although the manufacturers of optical fibers and endoscopes go to great lengths to minimize the thickness of the cladding relative to the diameter of the core, some portion of the cross sectional area of a fiber, and therefore of a fiber bundle, will be occupied by cladding and will therefore not be available to transmit light. In addition, light is also lost due to absorption and scattering of light within the fiber and reflective losses at the end faces. Some fiber bundles used in endoscopes use the same fibers to transport light in both directions.

A more common design, however, dedicates specific fibers within the bundle for illumination and others for light collection. This split design can significantly simplify the optics required at the proximal end of the bundle. Split fiber bundles have a significantly smaller effective fill factor than do those employing a common path and thus are less efficient in transmitting light between two locations.

The optics at the distal end of an endoscope (the end that is presented to the tissue being examined) are designed to image the tissue onto the end of the fiber bundle. The focal depth of these imaging optics dictates that the positioning and alignment of the distal end of the bundle relative to the tissue being examined be controlled within tight limits in order to ensure that the image presented at the proximal (or viewing) end of the bundle is in focus and is useable for measurement or imaging purposes. Quantitative measurements are particularly sensitive to the quality of focus.

Due to positional sensitivity, using an endoscope to make quantitative measurements requires considerable skill and excellent technique on the part of the operator. The alternative, forgoing the use of imaging optics at the distal end of the bundle and pressing the end of the bundle directly against the tissue, eliminates the depth of focus issue, but again requires considerable skill on the part of the operator to prevent the distal end of the fiber bundle from becoming contaminated by accidental tissue contact before it is abutted against the target area of the cervix. Such contamination can substantially interfere with the quality of the measurement.

Conflicting design requirements limit present endoscopes to sampling either the endo- or ecto-cervical region, but not both simultaneously. This limitation necessitates making two separate measurements using two separate devices in order to provide a complete cervical examination. The use of two devices does not, however, ensure adequate sampling of the transition region between the endo- and ecto-cervical tissues.

The cost of the fiber bundle used in an endoscope suitable for quantitative applications is sufficiently high that the fiber bundle must be reused in order to keep the cost per test within acceptable limits. This means that the bundle must be decontaminated before reuse to control infection and to ensure that adhering materials do not interfere with subsequent measurements. As decontamination is a time consuming procedure, a significant number of fiber bundles must be kept on hand to support the workflow of a screening site. The decontamination process can also cause both progressive and catastrophic damage to the fiber bundle leading to a relatively short useful lifetime before it must be replaced or repaired. In some cases, a disposable sheath is placed over the end of the bundle to prevent the bundle from coming into contact with the patient. Such a sheath can largely eliminate the need for frequent decontamination, but it can interfere with measurements made using the fiber bundle.

As is the case with all of the other major or system elements, endoscopes are large, expensive, delicate units that require considerable operator skill and attention. The net effect is that while tissue autofluorescence has been demonstrated to be capable of relatively rapidly detecting cervical abnormalities, the current embodiments of such systems are far too large, complex, delicate and expensive for widespread deployment as a routine screening tool.

Thus, a need remains for an efficient, economical system for in-vivo screening of cervical tissues. A need remains for a suitable replacement for the endoscope. A need remains for a screening system that employs exogeneous reagents to enhance cellular fluorescence.

### SUMMARY

The present invention utilizes an exogenous detection reagent to increase the signal level and suppress the background against which signal measurements are made. This, in turn, allows considerable simplification of the measurement instrumentation with concomitant reductions in size and cost.

Accordingly, the invention is found in an in-vivo tissue inspection device that includes an first non-imaging light collector that has an entrance and an exit and a second non-imaging light collector that has an entrance and an exit. The second non-imaging light collector is arranged so that its entrance is in light communication with the exit of the first non-imaging light collector. The device further includes a light guide and an optical element. The light guide is positioned between the second non-imaging light collector and the optical element.

The invention is also found in an in-vivo cervical tissue inspection system that includes a light source, a light detector, and the in-vivo tissue inspection device described hereinabove.

The invention is also found in a method of inspecting cervical tissue for abnormalities. The method includes contacting the cervical tissue with an exogenous fluorescent reagent that is preferentially taken up by abnormal cells, subsequently contacting the cervical tissue with light of a first wavelength, and detecting and measuring fluorescent light of a second wavelength. The light of a first wavelength and the fluorescent light of a second wavelength are both transmitted in a non spatially-resolved manner.

The invention is also found in a cervical screening method for screening cervical tissue that includes steps of applying an exogenous reagent to the cervical tissue, where the exogenous reagent is configured to cause abnormal cells to provide a discemable response to incident light, contacting the cervical tissue with an incident light sufficient to cause the discemable response to the incident light, where the discemable response includes emitted light of a particular wavelength, using a non-imaging light collector to gather and concentrate the emitted light, and impinging a detector with the gathered and concentrated light.

Other features and advantages of the present invention will be apparent from the following detailed description and drawings.

### BRIEF DESCRIPTION OF THE FIGURES

FIG. 1 is an illustration of a tissue inspection device according to a particular embodiment of the present invention.

FIG. 2 is an illustration of a tissue inspection and sampling device according to another embodiment of the present invention.

### DETAILED DESCRIPTION

The present invention replaces the fiber bundle used in present endoscopes with a simpler, less costly device that addresses the limitations of present technology. In particular, these limitations can be addressed by replacing the imaging fiber bundle and appurtenances with a device based upon the principles of non-imaging optics.

Optics

Non-imaging optics are optical devices that manipulate light in a non-spatially resolved manner. Such devices are distinguished by their simple structures and their exceptional efficiency in collecting light from one location and delivering that light to another location. The technology underlying such devices is extensively described in the open literature, most notably in the works of Winston. See, for example, U.S. Pat. Nos. 3,957,031; 4,002,499; 4,003,638; 4,230,095; 4,387,961; 4,359,265; 5,289,356; and 5,971,551, each of which are incorporated by reference herein.

The forms of non-imaging optical elements preferred in the present invention are known as compound parabolic and compound elliptical concentrators (CPC, CEC). These designations refer to the mathematical functions (parabolic and elliptical, respectively) that describe the shapes of these devices. The specific form selected for a particular embodiment of the present invention is primarily a matter of preference and convenience, and has minimal, if any, effect on the function or performance of the device. As such, these two base forms and their derivatives may be used interchangeably in the present invention. For simplicity, all following references will be to the CPC form with the recognition that alternative forms are equally suitable.

Mathematically, a CPC is a shape derived from the equation of a parabola having larger and smaller ends connected by a parabolic profile. The smaller end is usually called the throat of the device. Light enters the device through one end and exits through the other. Each end is characterized by an acceptance angle. Any light entering one end within the corresponding acceptance angle (less minimal reflectance and absorbance losses) is delivered to and exits from the other end. Light exiting the device is distributed over the entire acceptance angle of the exit end.

Since all light transiting the device passes through both end faces, the illumination density at the smaller end is greater than that at the larger end by an amount equal to the ratio between the areas of the two ends. The acceptance angle at each end of the device is determined by the relationship existing between the diameters of the ends of the device and the distance between these ends. Qualitatively, the smaller the axial ratio (length to diameter), the larger the acceptance angle. The present invention preferably utilizes each of these characteristics of a CPC.

In a preferred embodiment, the present invention is constructed around two CPC elements joined at the throat. The entry face of the device is intended to contact the cervical area to be examined. The diameter of the entry face of this composite device is defined by the diameter of the area on the cervix that is to be sampled while the length and throat diameter of the CPC comprising this portion of the device is largely a matter of design convenience. An axial ratio of about 3:1 and an area ratio of between 3:1 and 5:1 are preferred for this section.

Preferably, the throat diameter of the second CPC section is identical to that of the first section. It is desirable that the light exiting this second CPC section have a narrow angular distribution that matches the acceptance angle of the means used to deliver this light to the proximal end of the overall optical system. This is accomplished by selecting an axial ratio of between 5:1 and 10:1 in conjunction with a area ratio of approximately 2:1. The design particulars are illustrated in the Figures, which are described in detail hereinafter.

As the area of the exit face of this compound device is smaller than the entrance face, the optical power density at the exit face is greater than that at the entrance face by the ratio of the areas. This concentration effectively facilitates detection by increasing the signal levels.

The light exiting the second CPC section is delivered to optics at the proximal end of the system via a free space connection, a hollow core light guide, or an optical fiber. In these latter two cases, the diameter of the core of the light guide or fiber is selected to match that of the exit face of the second CPC. Note that the optical system as described is reversible in that light entering the system at the proximal end of the light delivery means will exactly retrace the path taken by light entering the system at the distal end of the device. This allows illumination and collection to be performed using the same optical path through the device.

The composite CPC used in this device can be fabricated by any of a number of established methods, selection between which is largely determined by whether the device is to be of the filled (immersed) or unfilled type. This, in turn, is largely determined by the overall length allocated to the CPC element during system design. A filled CPC generally has a lower axial ratio and, therefore, a shorter length than does an equivalent unfilled CPC.

Filled CPC's are most conveniently fabricated by casting or injection molding while unfilled devices are more conveniently fabricated by electroforming, casting or injection molding. Stamping, diamond turning and assembly from separately fabricated components are among the other methods that can be employed. Hollow core light guides are most conveniently fabricated by electroforming or by extrusion or tube drawing followed, in the case of a metallic guide, by electropolishing.

The present invention can, if desired, be configured to provide a limited degree of spatial resolution over the sampled area should this be desirable in a particular application. This is accomplished by assembling multiple CPC pairs, each of which has its own means of delivering light to the proximal optics. In this configuration, each CPC pair in the assembly contributes one spatially resolved point to a final measurement.

Such assemblies of CPC pairs are most conveniently fabricated as electroformed elements that may, if desired, be subsequently filled by a casting process. This level of spatial resolution is beneficial to the user in that it permits localizing a lesion to within a particular region of the cervix. This information facilitates follow-up procedures such as colposcopy, biopsy and therapy. In all cases, a compliant sleeve or collar projecting beyond the distal end of the CPC assembly facilitates alignment of the device with the cervix and serves as a shield to minimize the effects of stray light on the measurement.

The design of the CPC assembly can also be extended to accomplish simultaneous sampling of both the ecto-and endo-cervical regions. In this configuration, the distal end of the CPC is shaped to conform to the shape of the cervix with an extension that projects into the cervical canal. Preferably, the CPC is of the filled type and is designed to provide spatial resolution, preferably with one resolution element being dedicated to the canal and multiple elements being dedicated to the ectocervix. Filling the CPC allows for evanescent wave coupling into the canal and provides rigidity that assists in the insertion of the device into the canal.

Design details of the proximal optics are largely determined by the selection of the exogenous reagent. In particular, selection of the source of illumination, the wavelength selection means and, to a lesser extent, the detection means will be determined by the spectral properties of the particular reagent employed. In a preferred embodiment, a reagent such as BPD(Tm) having an excitation maximum in the vicinity of 630 nm and an emission maximum in the vicinity of 660 mn can be employed. Preferably, the fluorophore concentration will be determined from the ratio of emission intensities at 660 nm and 690 mn. In a preferred embodiment, tissue reflectance in the approximately 830-860 nm range can be used as a reference to correct for the hemoglobin concentration (hemoglobin absorbs light in the 630 nm range) and degree of oxygenation in the tissue being sampled.

Such an optical system could be constructed using the traditional epifluorescence/reflectance optical geometry. Such a geometry, which consists of an assembly of interference filters and dichroic reflectors is widely used in the prior art in those cases where excitation and emission share the same fibers in the fiber bundle. Alternative designs incorporating filter changers or Accousto-optic Tunable Filters, monochrometer or similar tunable wavelength selection means are also known in the prior art and are used in those instances where sequential rather than simultaneous measurements are acceptable. An “imaging spectrograph” geometry is also known and can be used. However, the limiting factor in each of these prior art embodiments is that they are large, complex and expensive, and in many cases suffer from low optical efficiency.

The present invention uses diffractive or holographic optical elements to accomplish these same ends. The differentiation between diffractive and holographic optics lies largely in the manner in which they are fabricated rather than in function or performance. For all practical purposes, a diffractive optical element is one that is fabricated using a largely digital process while a holographic optical element is fabricated using a largely analog process. Either type of optical element can be envisioned as combining the functions of a diffraction grating and a lens into a single structure.

In addition to combining wavelength selection and optical power (focusing) into a single integrated structure, diffractive and holographic optical elements also provide a means of precisely and selectively manipulating optical wavefronts. Specifically, diffractive and holographic elements can be made to transform any arbitrary wavefront incident on the entry aperture of the device into any other arbitrary wavefront at the exit aperture of the device. The specific wave front transformation(s) performed by a given device are determined by the details of its construction. One unique feature of such devices is that they can be constructed to perform multiple simultaneous independent transformations on the same incident wavefront.

In a preferred embodiment, solid state laser diodes emitting at 635 and 850 mn can be used as illumination sources. Moreover, it is preferred that fluorescence emissions from the cervix can be detected at 660 and 690 nm with bandwidths of 10 nm (Full Width Half Maximum) and that reflectance from the cervix at 850 nm can also be monitored. The 660 nm and 690 nm detectors are most conveniently blue enhanced silicon photodiodes or avalanche diodes while the 850 nm detector is most conveniently a gallium arsenide photodiode. Miniature photomultiplier tubes such as those available from Hamamatsu of Bridgewater, N.J., can also be used as detectors.

The diffractive/holographic optical element preferably performs several independent wavefront transformations. Specifically, the optical element should transform the wavefront associated with the light delivery means that connects the CPC to the proximal optics into narrowband wavefronts that match those of the two laser diodes and band limited wavefronts that can be efficiently coupled to each of the three detection elements. Furthermore, the optical element should spatially separate these various wavefronts in a manner that allows physical disposition of the light delivery means, lasers and detectors in the overall optical assembly.

The wavefront associated with the light delivery means can be described and modeled as that of light diverging from an extended, approximately circular source and illuminating the entire area of the optical element. The wavefronts associated with the lasers can be modeled as diverging elliptical beams from virtual point sources. As the divergence of such a beam is relatively small, they will illuminate only a portion of the optical element.

For convenience, the centroids of these beams will intersect the center of the element with the major axis of the two ellipses being at right angles to each other. The major constraint placed on the wavefronts incident on the detectors is that the shapes and sizes of the beams at the detectors match the shapes (square) and sizes (approximately 3 mm) of the respective detection elements. Dispersion in these beams also needs to be controlled in order to achieve the desired bandwidths. Ancillary interference filters may be placed at the entries to the detectors to further control the detection bandwidths.

In addition to these transformations, the optical element should rotate the plane of polarization of the 850 nm light by 45 degrees on each pass through the element, but should not affect the polarization of the light at the other wavelengths. Manipulating the polarization of the 850 nm light is preferable since excitation and detection of reflection is done at the same wavelength. Introducing the quarter wave rotation into the polarization of this light means that the plane of polarization of the reflected light will be rotated by 90 degrees relative to that of the light from the laser. Since the planes of polarization of the laser and reflected light are now orthogonal, the optical element can process each independently. This allows the 850 nm source and detector to be at physically separate locations.

A similar effect can be obtained by appropriate manipulations of the object and reference beams during the design and fabrication of the optical element. In those instances where a spatially resolved CPC is used, a separate optical element can be employed for each spatial channel or a single optical element can be constructed to process all of the channels. The use of a separate element per channel is preferred both to minimize interchannel crosstalk and due to the fact that the optical efficiency of such an element decreases and the cost increases as an increasing number of functions are integrated into a single structure.

Fabrication of a diffractive or holographic optical element that performs the functions described is accomplished by established means and methods that are well known to those skilled in the art. Fabrication services for such elements are available from a number of commercial sources. Implementing the proximal optics as a diffractive or holographic element allows the size, cost and complexity of these optics to be substantially reduced relative to what is possible using conventional optics.

The data reduction algorithms required in the present invention are rudimentary compared to those required by the prior art. In particular, the present invention requires one (or a small number of) ratiometric intensity determinations that have been corrected for tissue reflectance as determined using an third data channel. The prior art requires doing a very large number of spatially resolved measurements at high spectral resolution; deconvoluting the composite data to extract the signal changes of interest; and employing image analysis methods to localize the source(s) of the detected emissions.

The net effect is that the present invention requires substantially less computational power than is required by the prior art. This computing power can be provided using any of a rapidly increasing number of commercially available single board or “system on a chip” computers. Selecting such a computer that is packaged in a “credit card” or similar miniaturized format in conjunction with a miniature display and an embedded realtime operating system such as QNX a system offered by QNX Software Systems, Inc., Kanata, Ontario, Toronto, Canada allows the computer to be embedded in the hand held measuring device.

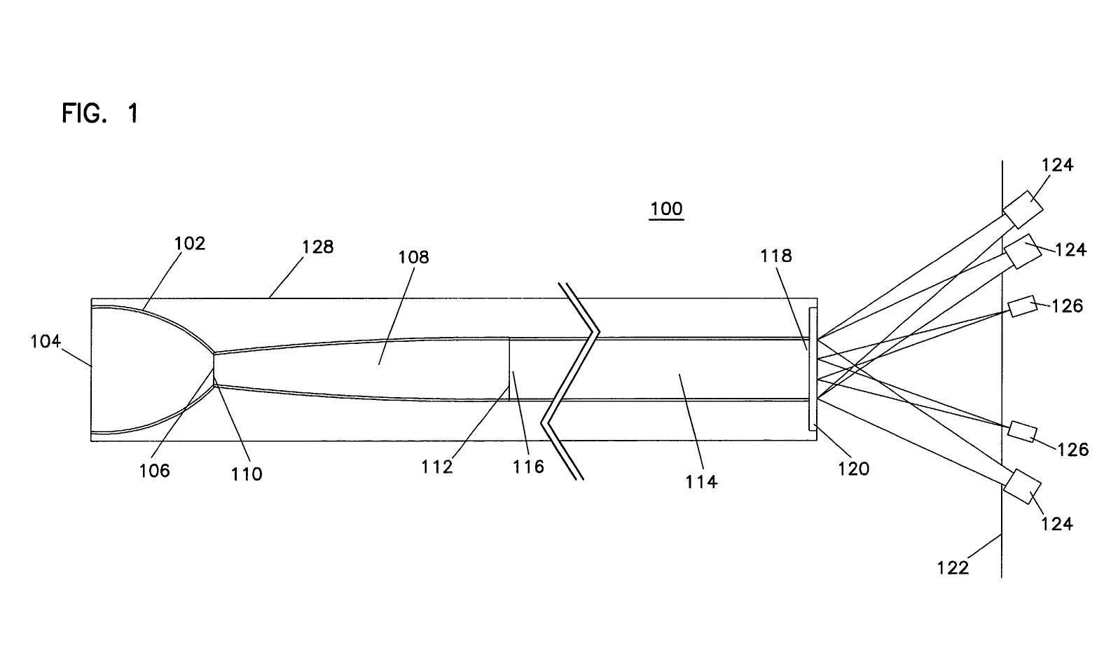

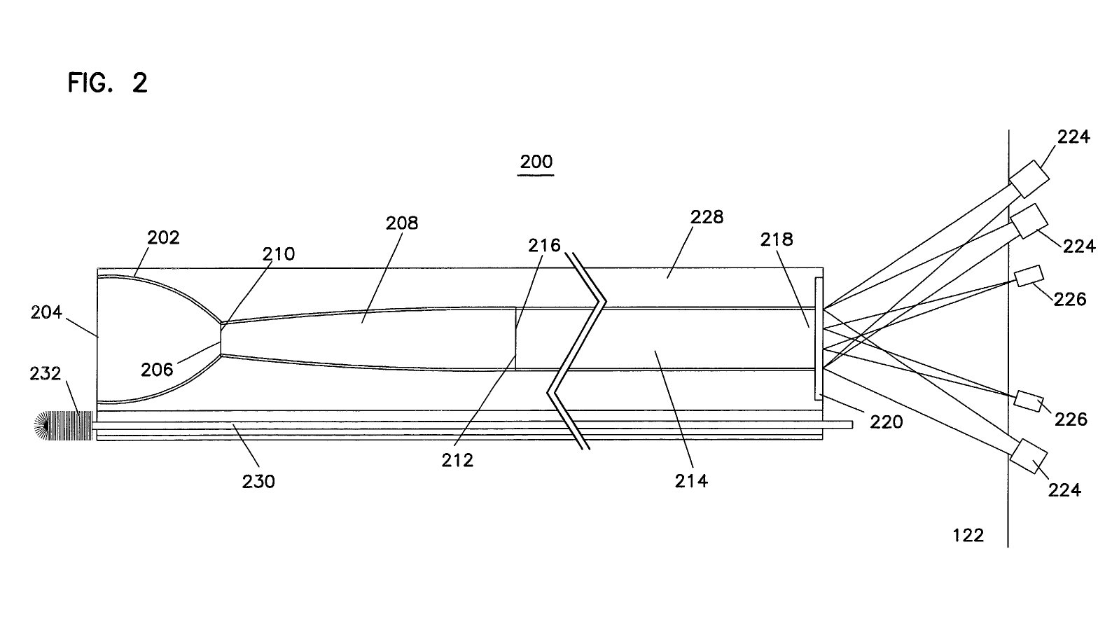

The Figures provide an illustration of several preferred embodiments of the present invention. FIG. 1 shows an in-vivo tissue inspection device 100 while FIG. 2 shows a particular embodiment of the present invention wherein in-vivo tissue inspection and sampling device 200 includes means to sample the tissue being examined.

In FIG. 1, the inspection device 100 is formed from a housing 128 that includes an entrance CPC 102 and an exit CPC 108. The first, or entrance, CPC 102 includes a first end 104 that is configured to contact the particular tissue to be sampled. While the first end 104 is illustrated as having essentially a flat or planar configuration, the invention is not limited to such. Indeed, the first end 104 can also be configured to match more closely with the profile of the tissue being examined. In a preferred embodiment, the tissue being examined is cervical tissue and the first end 104 can thus be configured to match a typical cervical profile.

The entrance CPC 102 also has a second end 106, which is also referred to as the throat of the CPC 102. The second end 106 of the entrance CPC 102 is preferably the same diameter as the first end 110 of the second, or exit CPC 108. The exit CPC 108 has a second end 112 that is preferably the same configuration and diameter as the first end 116 of the light guide 114. The second end 118 of the light guide 114 preferably contacts an optical guide 120.

FIG. 2 is quite similar, with the exception that the housing 228 further includes an elongate rod 230 that is attached to a biopsy apparatus 232. The biopsy apparatus 232 can be any suitable biopsy means known in the art, provided that it can obtain a tissue sample when desired. In a preferred embodiment, the biopsy apparatus 232 is a sampling brush 232 (as illustrated). In this embodiment, the physician or other health professional administering the screening test can, if desired, rotate and extend the elongate rod 230 so that the sampling brush 232 contacts the tissue being examined. Movement of the sampling brush 232 relative to the tissue causes tissue cells to be exfoliated and collected on the surface of the sampling brush 232. The sampling brush itself is described in greater detail in U.S. Pat. Nos. 5,999,844 and 6,081,740; each of which are incorporated in their entirety by reference herein.

The inspection device 200 includes an entrance CPC 202 having a first end 204 and a second end 206. As with FIG. 1, the entrance CPC 202 has a first end diameter that is significantly greater than the second end diameter. In contrast, the exit CPC 208 has a first end diameter (first end 210) that is not much smaller than its second end diameter (second end 212). As described in detail previously, the ratio between the first end diameter and the second end diameter defines the area ratio of the CPC while the axial ratio is a length to diameter indication.

In either embodiment, the inspection device 100, 200 has an optical element 120, 220 present at the proximal end 118, 218, respectively, of the light guide 114, 214. Preferably, the optical element 120, 220 is a diffractive optical element that can include only one diffractive or holographic element or can include a plurality of different elements. An aperture mask 122, 222 is used to direct and tighten the beams of light coming from the light sources 226 and to the detectors 224.

Reagents

One class of exogenous reagent preferably employed in the present invention is selected or derived from among the large number of chemical compounds that have been developed or evaluated as sensitizing agents for photodynamic therapy (PDT). These are fluorescent or fluorogenic compounds that are selectively and preferentially taken up, accumulated, and in the case of fluorogenic compounds, metabolized to form a fluorescent specie by abnormal cells.

In the intended therapeutic use of these compounds, exposing the target tissue to light of the appropriate spectral region will cause the compound that has been preferentially accumulated in abnormal cells to fluoresce. PDT therapeutic agents are designed such that the excited state produced upon exposure to light is highly reactive. This excited state reacts with water or other cellular constituents to produce “reactive oxygen” species such as singlet oxygen, hydroxyl radical or superoxide with a high quantum efficiency. These reactive oxygen species, in turn, react with other cellular constituents, thus damaging the cell to the point where it dies. As the PDT agent is selectively accumulated in abnormal cells, this provides a means of selectively killing abnormal cells in the presence of normal cells.

The present invention utilizes these PDT agents as detection, rather than therapeutic, reagents. As is the case of a therapeutic agent, a detection agent is selectively and preferentially taken up only by abnormal cells and is rapidly accumulated to high concentrations within these cells. Unlike the PDT therapeutic agents, the PDT detection agents are selected to efficiently produce high levels of fluorescence when optically excited at the appropriate wavelengths.

Where the excited state of a therapeutic agent is highly reactive and reacts with cellular constituents to form reactive oxygen species, the excited state of a detection agent is relatively unreactive and returns to its ground state via the emission of a photon. These detection agents are sometimes described as defective therapeutic agents because their output upon excitation is light rather than toxic chemicals.

Immunohistochemical and molecular probe reagents are another class of detection reagents that can be used in the present invention. These reagents incorporate a moiety such as an antibody (in the immunohistochemical reagents) or a molecule such as a lectin or a nucleic acid (in the molecular probe agents) that binds selectively to a preselected epitope or other molecular feature of a cell. These target features are selected from among those such as transferrin receptor, epidermal growth factor receptor or any of a wide variety of other cellular constituents whose presence or concentration has been correlated with the presence of the type(s) of cellular abnormalities of interest.

One or more “reporter” groups can directly or indirectly be coupled to the binding moiety to facilitate visualization. In the present invention, these reporter groups are most conveniently fluorescent species such as allophycocyanin, phycoerythrin, CY5 or the like, although fluorogenic, colored or chromogenic species may also be used. These reporter species can be selected so as to minimize the potential for interference with the measurements by tissue autofluorescence. To this end, the reporter species is preferably illuminated and quantitated at wavelengths greater than 550 nm. In addition to maximizing contrast, factors such as toxicity, photometric efficiency and speed of uptake are also considered during the selection process. Reagents of the types included in this class are well known to those skilled in the art.

Selecting a detection reagent according to these criteria results in abnormal cells having fluorescent emissions that are many times greater than those of normal cells and many times greater than tissue autofluorescence. Thus both the signal level and the signal to noise ratio are strongly enhanced over those observed with respect to tissue autofluorescence.

Additional benefits can be obtained by selecting the detection reagent such that the excitation and emission wavelengths do not significantly overlap those of tissue autofluorescence. For this reason, preference is given to reagents that excite and emit in the yellow, orange, red and near infrared spectral regions. Some of the PDT agents that have been found to be suitable for detection purposes include, but are not limited to: delta-aminolevulinic acid (ALA); Photofrin(Tm); BPD(Tm); Rhodamine 123; and a derivative of Nile Blue A developed by The Roland Institute for Science, Cambridge, Mass. Some suitable reporter moieties were identified above.

The means by which the exogenous reagent is administered to the patient depends upon the characteristics of the reagent and the intended application. Most PDT reagents are designed for injection because many of the target sites are not accessible for topical application. However, many PDT reagents including most of those listed above can be taken up by cells when applied topically. The immunohistochemical and molecular probe reagents are utilized in topical form. Some reagents are taken up rapidly upon topical application, but most require several hours to be absorbed or bound. Similarly, reagents such as ALA that must be metabolized in order to become active must be administered several hours before the measurements are to be made.

In the present invention, the reagent is preferably administered topically by applying a tampon or sponge containing the reagent to the cervix. Aspiration or spraying of the reagent onto the cervix can also be employed. In the instance of the tampon or sponge, one form of the applicator may be essentially as described in the U.S. patent application Ser. No. 09/603,625, which is hereby incorporated by reference herein; except that the face of the sampling element is made of a porous sponge material that serves as a reagent reservoir.

In the case of a slower acting reagent, the patient preferably inserts the device into her vagina several hours before a scheduled gynecological examination. Preferably, the sampling element rests against the cervix. This allows sufficient time for the reagent to diffuse from the sampling element and be taken up by any abnormal cervical cells that are present.

In those cases where a fast acting reagent is employed or where it was not practical or possible for the patient to apply the reagent to her cervix; where the patient did not comply with instructions to do so; or where the patient applied the reagent improperly, the reagent application can be performed by the clinician shortly before the examination. In this case, either a fast acting reagent is selected or the reagent is formulated to include an ingredient such as dimethyl sulfoxide that rapidly transports the reagent into the cells.

One limitation to the use of such transport agents to speed reagent uptake is that the ability of the reagent to discriminate between normal and abnormal cells is reduced. This approach is also not applicable to reagents that must be metabolized in order to become active. It is generally desirable for the clinician to remove any excess topical reagent from the cervix by washing or wiping before initiating the measurement procedure.

The uptake or binding of these detection reagents by cervical cells can be quantitated using the same instrumentation that is used to quantitate tissue autofluorescence. However, the substantially higher signal level and the selective concentration of the reagent in abnormal cells allows the performance requirements placed on the instrumentation to be relaxed somewhat relative to that needed for autofluorescence measurements. This can, in turn, somewhat reduce the cost and complexity of the instrumentation, but not to the level where it is practical for widespread deployment as a screening tool.

The present invention has been described with respect to using a single reagent or marker. However, the invention is not limited to such. Indeed, the present invention includes the use of a plurality of different markers that can be administered sequentially or simultaneously. In a preferred embodiment, the reagent actually includes a mixture of three different reagents or markers that are administered simultaneously.

While the invention has been described with reference to specific embodiments, it will be apparent to those skilled in the art that many alternatives, modifications and variations may be made. Accordingly, the present invention is intended to embrace all such alternatives, modifications and variations that may fall within the spirit and scope of the appended claims.

We claim:

1. An in-vivo tissue inspection device comprising:
an first non-imaging light collector having an entrance and an exit;
a second non-imaging light collector having an entrance and an exit, the second non-imaging light collector being arranged so that its entrance is in light communication with the exit of the first non-imaging light collector;
a light guide; and
an optical element, wherein the light guide is positioned between the second non-imaging light collector and the optical element.

2. The in-vivo tissue inspection device of claim 1, wherein the first non-imaging light collector and the second non-imaging light collector are each independently selected from the group consisting of a compound parabolic collector and a compound elliptical collector.

3. The in-vivo tissue inspection device of claim 1, wherein the first non-imaging light collector and the second non-imaging light collector are each independently selected from the group consisting of a filled non-imaging light collector and an unfilled non-imaging light collector.

4. The in-vivo tissue inspection device of claim 1, wherein the first non-imaging light collector has an axial ratio of about 3:1.

5. The in-vivo tissue inspection device of claim 1, wherein the first non-imaging light collector has an area ratio that is about 3:1 to about 5:1.

6. The in-vivo tissue inspection device of claim 1, wherein the second non-imaging light collector has an axial ratio that is about 5:1 to about 10:1.

7. The in-vivo tissue inspection device of claim 1, wherein the second non-imaging light collector has an area ratio of about 2:1.

8. The in-vivo tissue inspection device of claim 1, wherein the entrance of the first non-imaging light collector is sized in accordance with a particular tissue to be examined in-vivo.

9. The in-vivo tissue inspection device of claim 8, wherein the particular tissue to be examined comprises cervical tissue.

10. The in-vivo tissue inspection device of claim 8, wherein the cervical tissue to be sampled is one or more of endo-cervical tissue and ecto-cervical tissue.

11. The in-vivo tissue inspection device of claim 1, wherein the light guide is one of a free space connection, a hollow core light guide, or an optical fiber.

12. The in-vivo tissue inspection device of claim 1, wherein the optical element is one of a diffractive optical element and a holographic optical element.

13. An in-vivo cervical tissue inspection system, the system comprising:
a light source;
a light detector; and
the in-vivo tissue inspection device of claim 1.

14. The in-vivo tissue inspection system of claim 13, wherein the light source comprises a solid state laser diode.

15. The in-vivo tissue inspection system of claim 14, wherein the solid state laser diode emits at a wavelength that is at least one of about 635 nanometers and about 850 nanometers.

16. The in-vivo tissue inspection system of claim 13, wherein the light detector comprises a blue enhanced silicon photodiode or an avalanche diode, the light detector suitable to detect fluorescence emissions at wavelengths of about 660 nanometers and about 690 nanometers.

17. The in-vivo tissue inspection system of claim 16, further comprising a light detector comprising a gallium arsenide photodiode, the photodiode suitable to detect reflectance from a cervix at a wavelength of about 850 nanometers.

18. The in-vivo tissue inspection system of claim 13, further comprising a plurality of light sources and a plurality of light detectors.

19. The in-vivo tissue inspection system of claim 13, wherein the light detector comprises an imaging array detector.

20. The in-vivo tissue inspection system of claim 13, further comprising a source of an exogenous reagent to enhance cellular fluorescence.

21. The in-vivo tissue inspection system of claim 13, further comprising an external housing comprising a sampling element.

22. The in-vivo tissue inspection system of claim 21, wherein the sampling element comprises a biopsy apparatus that can be manipulated to exfoliate and collect cervical cells.

23. A method of inspecting cervical tissue for abnormalities, the method comprising steps of:
contacting the cervical tissue with an exogenous fluorescent reagent that is preferentially taken up by abnormal cells;
subsequently contacting the cervical tissue with light of a first wavelength; and
detecting and measuring fluorescent light of a second wavelength;
wherein the light of a first wavelength and the fluorescent light of a second wavelength are both transmitted in a non spatially-resolved manner.

24. The method of inspecting cervical tissue of claim 23, wherein the step of contacting the cervical tissue with an exogenous fluorescent reagent comprises application with one of a tampon, a sponge, a wipe or a brush.

25. The method of inspecting cervical tissue of claim 23, wherein the step of contacting the cervical tissue with an exogenous fluorescent reagent comprises application via one of aspiration and spraying.

26. The method of inspecting cervical tissue of claim 24, wherein the step of transmitting the light of a first wavelength and the fluorescent light of a second wavelength in a non-spatially resolved manner comprises transmitting the light of a first wavelength and the fluorescent light of a second wavelength through a non-imaging optical device.

27. The method of inspecting cervical tissue of claim 23, wherein the step of contacting the cervical tissue with the light of a first wave further comprises contacting the cervical tissue with light of a plurality of distinct wavelengths.

28. The method of inspecting cervical tissue of claim 27, further comprising a step of measuring reflectance of light of a particular wavelength selected from the plurality of distinct wavelengths.

29. The method of inspecting cervical tissue of claim 23, wherein the exogenous fluorescent reagent is selected from the group consisting of a photodynamic therapy reagent, an immuno-histochemical reagent, and a molecular probe.

30. A cervical screening method for screening cervical tissue, the method comprising steps of:
applying an exogenous reagent to the cervical tissue, the exogenous reagent configured to cause abnormal cells to provide a discemable response to incident light;
contacting the cervical tissue with an incident light sufficient to cause the discernable response to the incident light, the discernable response comprising emitted light of a particular wavelength;
using a non-imaging light collector to gather and concentrate the emitted light;
and
impinging a detector with the gathered and concentrated light.

31. The cervical screening method of claim 30, wherein the step of using a non-imaging light collector comprises using at least one compound parabolic collector.

32. The cervical screening method of claim 30, wherein the step of using a non-imaging light collector comprises using at least one compound elliptical collector.

33. The cervical screening method of claim 30, wherein the step of impinging a detector comprises using an optical element comprising a diffractive optical element in order to polarize the gathered and concentrated light.

34. The cervical screening method of claim 30, wherein the step of contacting the cervical tissue comprises passing the incident light through a non-imaging collector.

35. The cervical screening method of claim 30, wherein a filled non-imaging collector is used to screen cervical tissue comprising endo-cervical tissue.

36. The cervical screening method of claim 30, further comprising a step of obtaining a sample of the cervical tissue in response to an indication of abnormality.
